(19) United States
(12) Patent Application Publication
(10) Pub. No.: US 2025/0031689 A1
(43) Pub. Date: Jan. 30, 2025

(54) CRYOPRESERVATION COMPOSITIONS AND METHODS

(71) Applicant: Deepak Agrawal, Oro Valley, AZ (US)

(72) Inventor: Deepak Agrawal, Oro Valley, AZ (US)

(21) Appl. No.: 18/764,297

(22) Filed: Jul. 4, 2024

Related U.S. Application Data
(60) Provisional application No. 63/515,187, filed on Jul. 24, 2023.

(51) Int. Cl. A01N 1/02

(52) U.S. Cl. CPC A01N 1/0221

(57) ABSTRACT

Compositions and methods for cryopreservation including an amino acid at a concentration at, for example, between 50 mM and 1 M. Glutamate alone or combined with other amino acids and/or other cryoprotective molecules provides cell protection against freeze damage. Moreover, compositions and methods for cryopreservation may be provided with less or minimal toxicity by including one or more cryoprotectants at a concentration that is insufficient for crypopreservation of cells when used at that concentration alone.

### BACKGROUND

The field of the embodiments disclosed herein relates to cryopreservation and more particularly to the freezing with minimal accompanying loss of viability or functionality of a living cell and cell derived or composed materials such as tissues and organs.

Cryopreservation of living systems has been actively pursued for over 100 years, from viruses and bacteria to plants and animal cells. Although human interest may extend back to the first observation of a thawing insect or plant in a subzero geography, experimental investigations into this process were actively pursued subsequent to the innovations in refrigeration of the mid-1800's. One of the first reports of specific cryoprotectants and their activity (Keith, 1913) identified glycerol as a cryoprotectant for E. coli. Almost as significantly, it may have been the first to reveal that inhibiting ice formation, or solidification, was a key in the protective effect through a freeze-thaw cycle. The latter remains true to the present, being the primary mechanistic effect through which cryoprotectant action is believed to act. Beyond the initial demonstration with E. coli, glycerol was the first true cryoprotectant for mammalian systems, including fowl spermatozoa (Parkes, 1945), red blood cells (Mollison et al, 1952) and both mouse L cells and human Hela cells (Scherer and Hoogasian, 1954). Soon after the demonstration of successful cryopreservation of mammalian cells using glycerol, it was found the ice inhibiting molecule Dimethylsulfoxide, DMSO, was a very effective cryoprotectant (Lovelock and Bishop, 1959). Its efficacy is borne out by over half a century of investigation, as it remains the dominant cryoprotectant across a wide range of biologyical applications, preferred in almost all applications despite its known toxicities.

So while DMSO mixed in a variety of formats, from simple phosphate buffered saline (PBS) buffers to mixes of various growth medium containing both animal derived and recombinant proteins and serum as well as other types of ancillary molecules, is an effective cryoprotectant, toxicity can limit its application. Thus, a current need driven largely by cell therapy markets is to mitigate toxicities or adverse events related to DMSO. Moreover, there remains the desire to identify modalities that could effectively substitute for DMSO while minimizing the associated toxicities.

### SUMMARY OF THE DISCLOSED EMBODIMENTS

One of the underlying issues in replacing DMSO as a cryoprotectant may lie in an incomplete understanding of the details of how it is able to provide for cell recovery. A more compete mechanistic understanding might allow for designing rational approaches for alternate molecule(s).

An examination was made of the experimental similarities of the class of molecules identified as cryoprotectants, that they are utilized at supraphysiological concentrations sufficient to induce osmotic stress and cell dehydration, and that the compatibility of these particular solutes provides tolerance to high concentrations to permit inhibition of ice formation as the temperature approaches subzero targets. Further work leveraged the correlation between these features and their cryoprotective capacities to potentially identify and target molecules that were not known to have this high concentration induced cryoprotectant activity.

One class of molecules that has remained largely unstudied in this regard are amino acids. With the exception of proline and betaine, effects of amino acids in cryopreservation at these concentrations have largely been unstudied to this point. Proline and betaine have both been shown to effective for cryoprotection, but only when present intracellularly at supraphysiological levels, requiring specific cell loading through prolonged incubation or by electroporation (Gao, 2021), which itself can be damaging to cells. Amino acids have been studied in multiple polymeric forms including antifreeze peptide templated variants and homopolymeric peptides of charged residues. However, surveys of free amino acids in the context of driving osmotic shock and subsequent metabolic events that could provide both intra-and extracellular cryoprotection, have not been reported.

In some embodiments, compositions and methods have been discovered whereby a strong cryoprotectant activity across three different mammalian cell lines was achieved utilizing glutamate. As demonstrated herein, it is active as a sole agent in this regard, but can also act as a primary cryoprotectant in a number of buffers, including growth medium and physiological saline, demonstrating its versatility in a way that most primary cryoprotectants have been utilized. It requires hypertonic/supraphysiological concentration ranges to be effective for cryoprotection as a sole agent.

In further embodiments, and unlike other cryoprotectants, we also describe compositions and methods in which one or more additional amino acids can be substituted for glutamate to achieve hypertonic mixtures as long as there is a minimal level of glutamate present. Perhaps not surprisingly, some of the amino acid mixtures had cryoprotective activity in the absence of glutamate, though not as effective.

In still other embodiments, compositions and methods are disclosed in which glutamate can be used in the presence of other cryoprotectants such as DMSO, and glutamate was found to greatly increase the protection afforded by very low concentrations of DMSO, suggesting these molecules may be synergistic in their action

The idea of utilizing a chemistry based entirely on amino acids to create a cryoprotectant solution has many potential advantages. The most compelling being, perhaps, that any resulting composition would be made entirely of molecules in the natural milieu of cells. By virtue of the fact that my studies revealed inclusion of a wide variety of amino acids with glutamate, including those with polar and charged side chains, it is possible that acclimation of different cell lines or types could be done by biasing the amino acid composition to one that might be more compatible with that cells particular contextual biases based on its own biochemical makeup. Additionally, the identification of an amino acid chemistry able to confer cryoprotection that is compatible with growth media, physiological buffers and molecules, and other cryoprotectants also creates the opportunity to create compositions that also include molecules and other chemistries that can promote cell potency, growth, or stability.

Moreover, embodiment herein reveal single amino acids that act synergistically with low (e.g., 1%) DMSO concentrations to bring about effective cryoprotection. Furthermore, amino acid mixtures also have been found to exhibit enhanced synergy in the presence of low concentrations of DMSO.

### BRIEF DESCRIPTION OF THE DRAWINGS

FIG. 1 illustrates the cryoprotective ability of glutamate solutions for HEK293 cells.

FIG. 2 depicts the ability of glutamate to impart cryoprotective ability to cell culture medium with HEK293 cells.

FIG. 3 shows the ability of glutamate to impart cryoprotective ability to cell culture medium with HCT-115 cells.

FIG. 4 illustrates that glutamate, both alone and in mixtures with other amino acids, serves as a cryoprotective agent.

FIG. 5 reveals compatibility between glutamate and known cryoprotective agents betaine and trehalose.

FIG. 6 reveals synergy of glutamate with very low concentrations of DMSO and glycerol.

FIG. 7 reveals single amino acids that act synergistically with low DMSO concentrations to bring about effective cryoprotection.

FIG. 8 reveals amino acid mixtures exhibit enhanced synergy in the presence of low concentrations of DMSO.

### DETAILED DESCRIPTION

Investigations of cold hardiness in plants and insects (Sakai, 1962, Salt, 1961) provided the basis for early discoveries in animal cells, with sugars and sugar alcohols, including glycerol, trehalose, and sorbitol supporting freeze-thaw survival, in some cases to temperature below liquid nitrogen. The range of investigation and the large number of approaches generated from these and other cold tolerant systems and the currently continued preference for DMSO highlight the efficacy of the latter. A curated compilation of molecules exhibiting cryoprotective activities was first published by Karow (Karow, 1969). This number was later reduced to 25, of which only 10 were identified as generally effective for cryoprotection (Ashwood-Smith, 1987). Although serum, dextran, polyethylene glycol (PEG), sucrose, among others were identified with some cryoprotective activity, only DMSO and glycerol were noted as very effective and commonly used, the others less so.

A more recent update (Elliot et al, 2017) provided curation including effectiveness for a slightly larger list of 28 compounds, separated into five classes based on molecule type: alcohols, sugars and sugar alcohols, polymers, sulfoxides & amides, and amines. Again, most notable are the two most effective categories in this work: i) highly effective across cell types and ii) very effective for defined cell types only. Both included just one molecule each: DMSO and glycerol, respectively, suggesting broad efficacy and utility across many cell models to have maintained class leading status for 60+ and 110+ years. More recently, a great deal of effort and progress has been made in the area of polymers, both synthetic and biological, in the form of oligomeric ampholytes such as amino acid derivatives and biologics based on antifreeze protein templates.

It is important to specify the context of the above discussion of cryoprotectants includes only those molecules that have been found to impart significant cryoprotectant activity without the aid of additional chemical species, i.e. cryoprotectants are capable of sustaining viability through a freeze-thaw cycle at subzero temperatures in the absence of other molecules. A number of additional molecules have been tested for their ability to influence overall viability or other desired character in the revived population. Typically these molecules are not obligatory and the extent to which many affect the cryopreservation process can be represented by fractional improvements in post-thaw viability. The most well studied example of such a molecules may be betaine and proline, which have been demonstrated to increase freeze-thaw viability in the case of a number of systems, including spermatozoa [Li 2003], red blood cells [RBCs, Dou 2019], and oocytes [Zhang, 2016].

Well studied across a variety of animal models, including goat, ram, and donkey, the work done with Cygnus monkey spermatozoa [Li, 2003] examined several amino acids, proline, glutamine, and glycine, demonstrating a modest increase with each, approximately 20% above that obtained with the control, at 5-10 mM, but interestingly revealed a decrease in post-thaw recovery beyond 10 mM, revealing a loss of improvement and further decline at 50 mM, the highest concentration tested. These studies largely mirror the studies of spermatozoa in other animals, where it is typically used at similar or even lower concentrations to effect similar increases in recovery. However, in no case was proline ever tested or found to be effective as a sole cryoprotectant at any concentration. A study of mouse oocytes (Zhang, 2016) demonstrated an increase in implantation rate of 6-10% by the addition of 2M proline. However, as above, these increases were measured in the presence of two additional established primary cryoprotectants: DMSO and ethylene glycol. There was no activity reported for the ancillary proline in the absence of the additional bona fide cryoprotectants. Finally, in the case of RBCs [Dou, 2019], 1.5M proline was able to reduce hemolysis of frozen cells, but only in the presence of trehalose. The improvement was unclear given the low glycerol concentration used in positive controls supported high levels of hemolysis.

A second class of ancillary molecules is exemplified by their ability to scavenge free oxygen radicals and protect from oxidative damage (Alvarez and Storey, 1992 and Limaye, 1997). Early studies following the identification of adverse effects of free radicals on recovery from freeze-thaw revealed a number of molecules able to mitigate this damage and offer improved yield including ascorbate, catalase, alpha-tocopherol, and glutathione (Limaye, 1997). A host of additional molecules have been examined since then in various cell models. Like other ancillary molecules, these antioxidants are without prima fasciae activity as cryoprotectants, but can facilitate or improve recovery and yield in a cell type specific manner. A last example of ancillary molecules might also include a number of polymers such as hydroxylethyl starch or polyethylene glycol that, while reported as having “cryoprotective” activity without direct evidence in the absence of another cryoprotective agent, facilitate the activity of one of the bona fide cryoprotectants listed above.

In fact, distinguishing the mechanisms of actions for all of the molecules listed above becomes challenging given the relative resolution at which today's theories account for cryopreservative activities. The consensus view, as mentioned above, retains the common understanding and basic feature identified in 1913 that ice crystallization and its impact on cell integrity is a primary component of cryoprotection through which all of the primary cryoprotectants listed above bring about their protection against a freeze-thaw cycle. The proposed mechanism of action for all of these molecules typically resides in an ability to inhibit ice formation, through either nucleation, recrystallization, or both, at high sub-zero temperatures where much of the damage of ice formation is believed to take place.

Thus one manner to refer to compositions conferring cryoprotection on mammalian cells is the utilization of (a) one or molecules, that will typically act in a colligative manner at high concentration to induce osmotic stress and dehydration, to inhibit ice formation and other necessary activities, and (b) optionally one or more ancillary molecules that acts to improve one or more aspects of yield as measured by one or more assays that describe viability or a functional aspect of the cell. This definition represents much of the consensus understanding of cryopreservation, and likely the vast majority of cryopreservation buffers in use, in this context. To meet the requirement of (a), a primary cryoprotectant requires tolerance to high concentration, at least in the workflow timeline, it does not require a complete absence of toxicity as exemplified by DMSO.

However, as outlined above, despite a significant number of studies and resulting strategies for preserving cells undergoing a freeze-thaw cycle, none have provided the overall efficiency and broad applicability of DMSO.

For the studies detailed below, the following procedures were used for preparation of cell cultures prior to cryopreservation: Both HEK-293 and HCT-115 cells were cultured in DMEM medium supplemented with 10% calf serum or 10% fetal calf serum. Two or three days prior to initiating a cryopreservation study, cells were seeded onto 100 mm tissue culture treated dishes in the above described culture medium and placed into a humidified, 5% CO₂ incubator and allowed to propagate for 2 or 3 days. Preparation of growing cultures for cryostorage was performed as follows: Medium from 100 mm dishes containing growing cultures prepared as described above was aspirated and replaced with saline containing 0.5 mM EDTA and allowed to incubate for 2-3 minutes. This material was also aspirated and replaced with saline containing 0.25% trypsin and 0.25 mM EDTA. After incubating this second mixture for an additional 3-5 minutes and observing partial detachment of cells from dishes, cells were removed by using inertial force against the dish or by using a stream of saline or base culture medium to dislodge and collect cells from the dish. Cells harvested in this manner were separated by centrifugation (100 g×3 min), resuspended in the desired cryostorage solution, dispensed into polypropylene vials, positioned in microtube racks that were placed into a −80° C. freezer. Cells were kept frozen for 24-168 hours, at which time cryovials were removed to wet ice and cells thawed and plated into appropriate tissue culture vessels, typically 6 or 12 well plates. Cells were visualized with the aid of a Nikon TE300 microscope with an attached camera to allow documentation.

As described above, we wished to test the ability of amino acid species to serve as primary cryoprotectants per the definition provided, i.e. at concentrations at which they provided osmotic pressure sufficient to induce cell dehydration. For this purpose we prepared solutions of several amino acids, including alanine, glycine, proline, glutamate, histidine, aspartate, leucine, lysine, phenylalanine, threonine, arginine, and serine. These were standardized to 1M and adjusted to pH 7.1-pH 7.3, where possible or to maximal concentrations when 1M was above the solubility such as histidine and leucine. These solutions were used by dilution in water to test for their ability to serve as a cryoprotectant or diluted to the desired target concentration in a variety of base media including saline, and the two growth mediums described above (DMEM and OptiMeM) in the studies with three different mammalian cell lines described below described below.

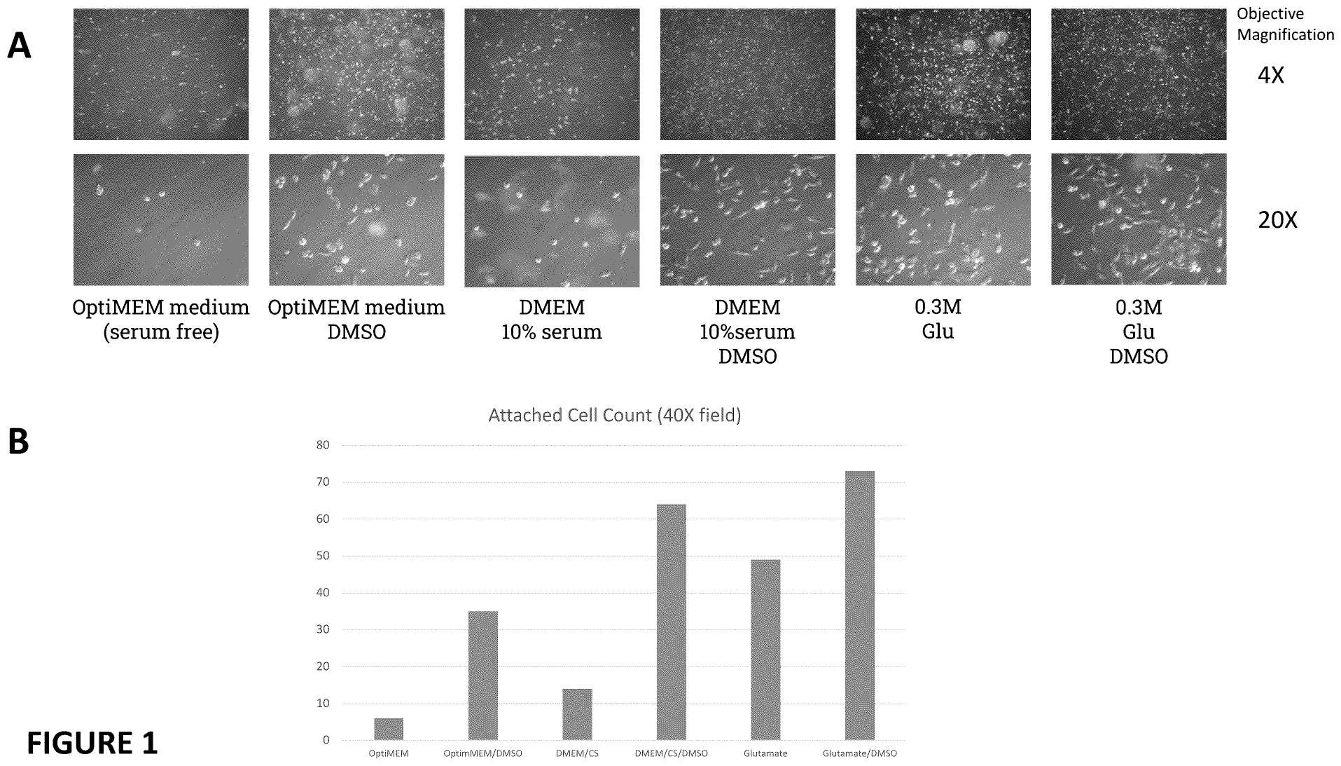

FIG. 1. Glutamate is a potent cryoprotectant as a sole reagent. An initial survey revealed cryoprotective capacity of several amino acids, but among several tests, glutamate appeared to consistently exhibit the highest degree of post-thaw viability when testing with HEK293 cells. To test this capacity against a well-established cryoprotectant from those described above, HEK293 cultures were prepared as above in one of three base cryostorage mediums as noted below each pair of photographs depicted in panel A: (a) OptiMEM culture medium (ThermoFisher), a serum free growth medium, (b) DMEM culture medium containing 10% serum, or (c) 0.3M glutamate pH 7.2. One sample was prepared in each storage medium as described (labels on the bottom of each vertical pair of photographs), and an additional sample for each was prepared with 7.5% DMSO. 23 hours after plating thawed cells, cultures were visualized with 4× (upper) and 20× objectives (lower). It can be seen from these results that attachment of cells at 23 hours requires DMSO for either cell culture medium, as cells in either growth medium absent DMSO fail to attach, while those in samples containing DMSO do attach. A comparison of glutamate as a sole cryoprotectant at 0.3M is able to provide attachment at least to the level of either medium containing DMSO. We also tested addition of 7.5% DMSO to 0.3M and found modest increases, suggesting that these may be compatible cryoprotectants as well. The images were also analyzed using NIH Image to count cell numbers, depicted in panel B.

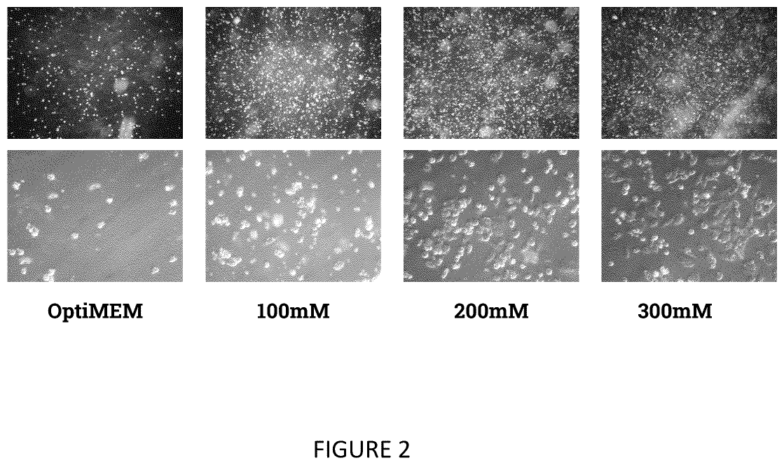

FIG. 2. Cryoprotectant activity of glutamate can be imparted to cell growth medium. HEK293 cells were prepared as described above in OptiMEM medium and apportioned into 4 samples containing 0 mM, 100 mM, 200 mM, or 300 mM glutamate (labels below photograph). After completing a freeze-thaw cycle, each sample was plated and visualized 23 hours after plating. It can be seen from these results that glutamate at the lowest concentration provides some extended post-thaw viability at 100 mM, but at 200 mM and 300 mM, not only is there increased viability, but the cells are able to metabolize sufficiently to attach to the substrate to increasing degree. The cells stored in 0.3M glutamate clearly demonstrate improved entry into the growth cycle as compared to the cells in medium alone. Clearly this demonstrates not only can glutamate serve as a sole cryoprotectant, as shown in FIG. 1, but it has the ability to impart its cryoprotective ability to render a composite solution such as optimum to become a cryoprotectant solution in the absence of serum or any additional ingredients.

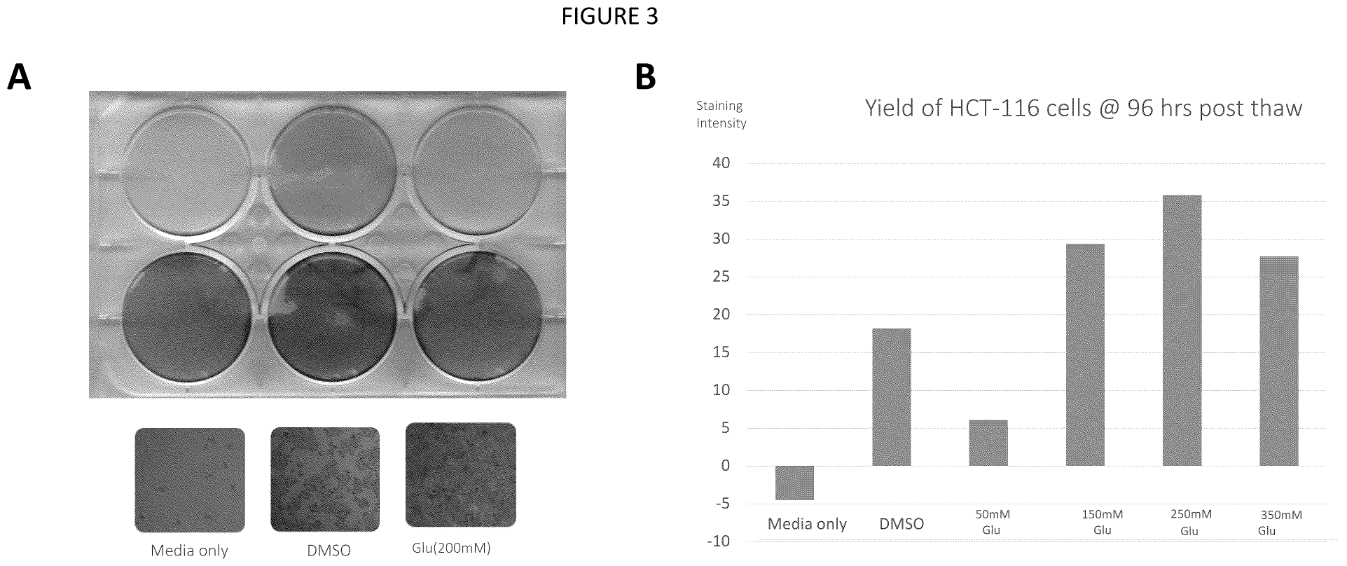

FIG. 3. Cryoprotectant activity of glutamate requires concentrations greater than 50 mM. The dose response relationship of glutamate and its ability to impart cryoprotective activity to OptiMeM prompted the question of our findings being extensible to other cell types such as HCT 115 cells (FIG. 2). Cells were prepared as described above in OptiMEM medium containing either no additions, 7.5% DMSO, 50 mM glutamate, 150 mM glutamate, 250 mM glutamate, or 350 mM glutamate. Thawed cells were plated, washed at 24 hours to remove debris, and allowed to grow for a total of 96 hours, at which time they were fixed with 10% formol (15 minutes). Fixed cultures were stained with 0.2% crystal violet for 2 hours, destained in 4 changes of distilled water, dried and scanned on an Epson scanner at 1200 dpi (panel A). The density of stain from each culture, corresponding to cell yield, was quantified using ImageJ (NIH). The density of each culture was plotted in a bar graph and is shown in panel B. The stained 6-well plate containing each culture is shown in panel A, with insets showing a photograph from representative cultures visualized with a 4× objective after staining. The order of the samples in the plate shown in panel A is (left to right, top to bottom): media alone, media+7.5% DMSO, media+50 mM glutamate, media+150 mM glutamate, media+250 mM glutamate, media+350 mM glutamate. The data reveal that glutamate is a cryoprotectant that can work as such across cell lines and can also work as a sole reagent or can be used in conjunction with other physiological buffers or growth mediums.

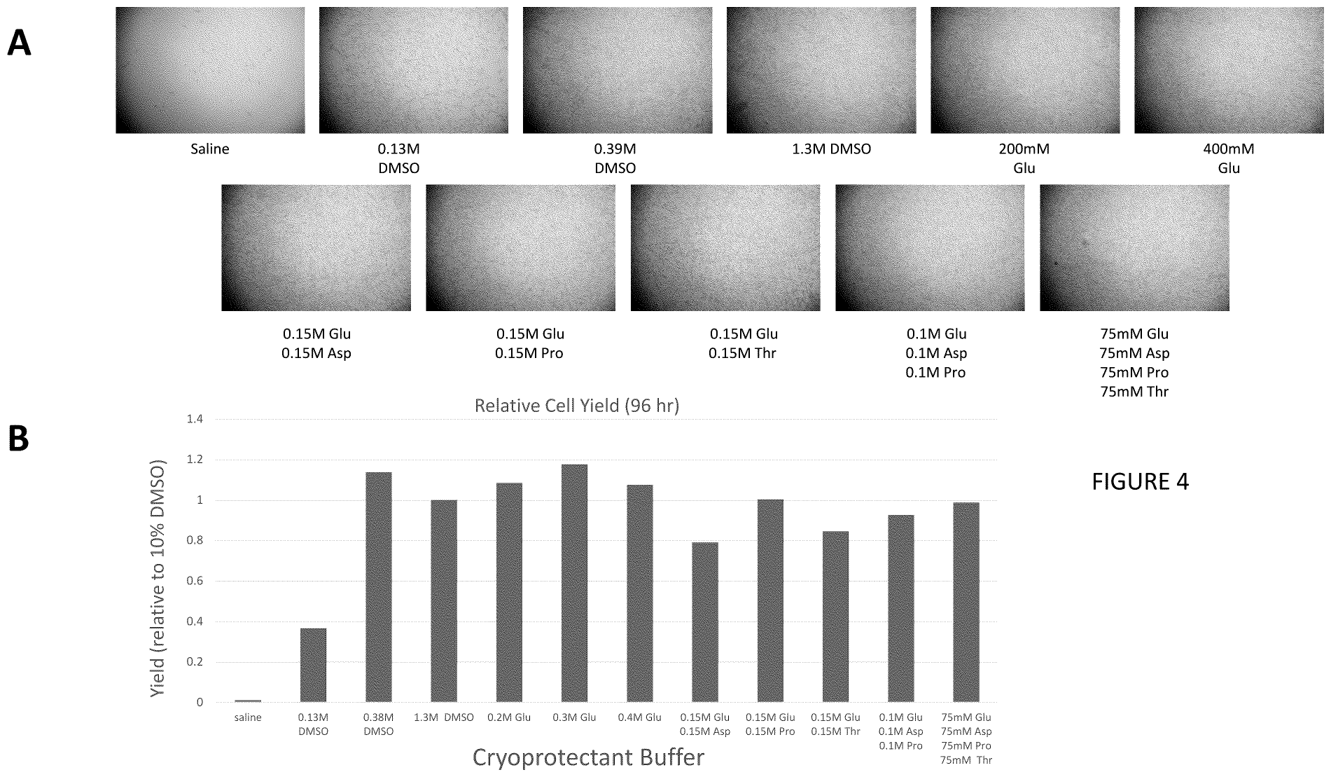

FIG. 4. Glutamate, both alone and in mixtures with other amino acids, serves as a cryoprotective agent. Although we found glutamate to serve as a cryoprotectant alone, the workflow for freezing cells requires that the cryoprotectant solution be used directly to resuspend centrifuged cell samples. We developed a workflow that tested cryoprotection of cells suspended in physiological saline (150 mM NaCl), overcoming the need to resuspend centrifuged cells directly in a final cryoprotection buffer. We reasoned this approach would allow for multiple comparisons with a single cell sample. Thus, in these studies, trypsinized cells were centrifuged as described above, but resuspended in saline prior to diluting into a test solution. The photographs in FIG. 4A depict representative fields at 4× magnification of 6-well plates seeded with ovine pre-adipocyte cells, frozen in solutions comprised of agents and concentrations indicated to the left of each row. The cells in each field were enumerated by using ImageJ (NIH) to carryout noise reduction and thresholding before enumerating cell particles (FIG. 4B). It should be noted that dilution of the initial saline sample contributed 0.105 M NaCl concentration in addition to the agents indicated to the left. To provide a positive sample, we also tested DMSO at three concentrations (1%, 3%, and 10%). The results clearly demonstrate the lack of survival after storage in saline at −80° C., and that DMSO serves as a capable cryoprotective agent at both 3% and 10%. Surprisingly cryoprotective activity was apparent even at 1% DMSO, albeit much less than was observed at higher concentrations. Glutamate as a sole agent provided significant cryoprotection, as measured by cell yield, comparable to the higher concentrations (3% and 10%) of DMSO. Our earlier studies suggested cryoprotective activity may be present in several amino acids and might provide a path to creating cryoprotectants composed of amino acid mixtures. Therefore, we also tested binary mixtures as well as a ternary and a quaternary mixture. A binary equimolar mixture of glutamate with either asparate, proline, or threonine were tested at 0.3M, revealing all had the capacity to provide cryoprotection to different extents. Furthermore, the use of either a ternary mix (Glu, Asp, Pro) or quaternary (Glu, Asp, Pro, Thr) revealed that these amino acids could be mixed in equimolar amounts to reduce the concentration of any one amino acid, while retaining the ability to support cryopreservation. This demonstrates the potential utility of using amino acid mixtures for cryoprotection. In the example shown, glutamate concentration was reduced to 75 mM while retaining a similar level of freeze-thaw protection.

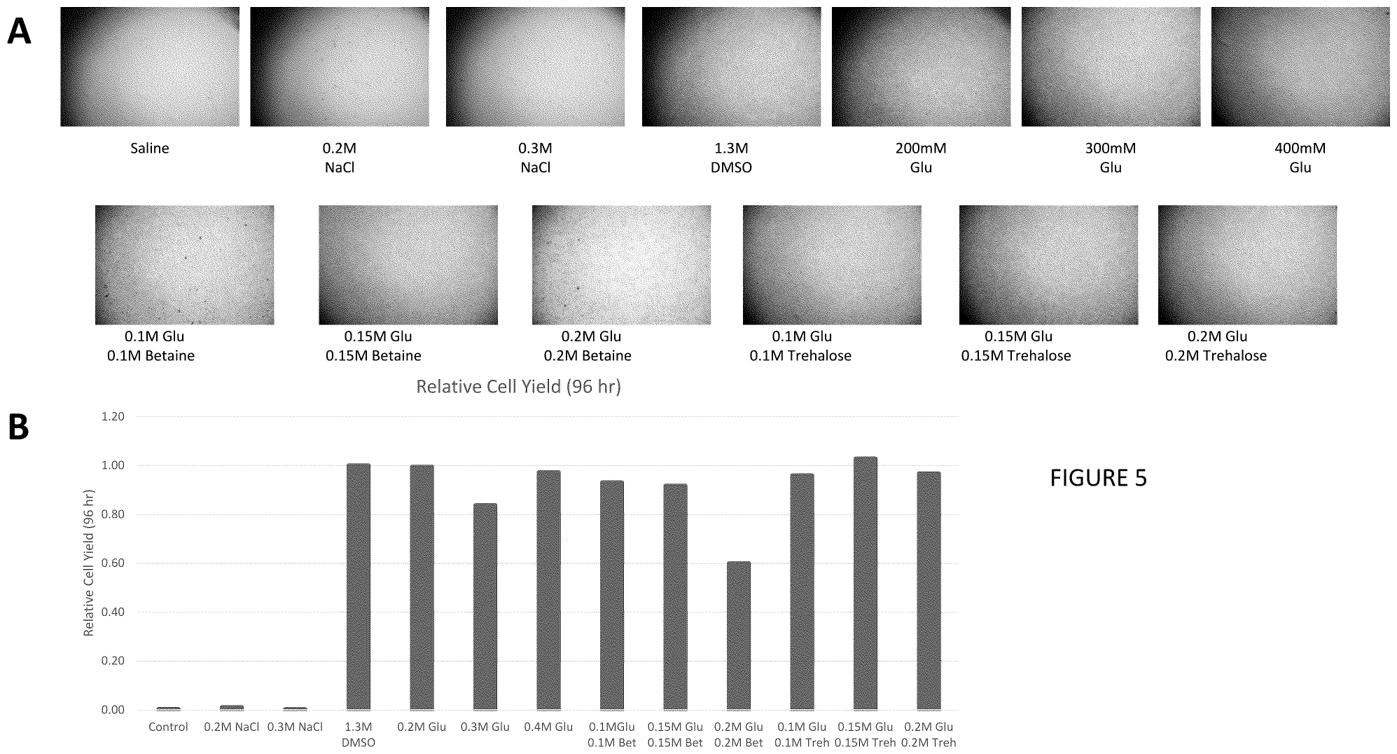

FIG. 5. Compatibility of glutamate and known cryoprotective agents betaine and trehalose. The previous study revealed the cryoprotective activity may be obtained with one of several amino acids at sufficient concentration. The concentration requirement is capable of being met either with a single appropriate amino acid, or a combination of appropriate amino acids. We ascertained if such synergy might also exist with other well established cryoprotectants such as betaine or trehalose. In a study similar to the one described in FIG. 4, equimolar solutions of glutamate with either betaine or trehalose were used at multiple concentrations and compared to their ability to promote cryoprotection in comparison to DMSO or glutamate (FIG. 5). The results, shown in FIG. 5A and quantified in FIG. 5B, reveal that equimolar mixtures of glutamate with either betaine or trehalose can be used for cryoprotection, albeit the combination of glutamate and betaine appears less effective at higher concentration. It also demonstrates that simply increasing ionic strength by addition of NaCl at concentrations where glutamate acts as a CPA does not provide any significant cryoprotection. Thus, glutamate appears to be compatible with a variety of additional cryoprotectants including betaine and trehalose.

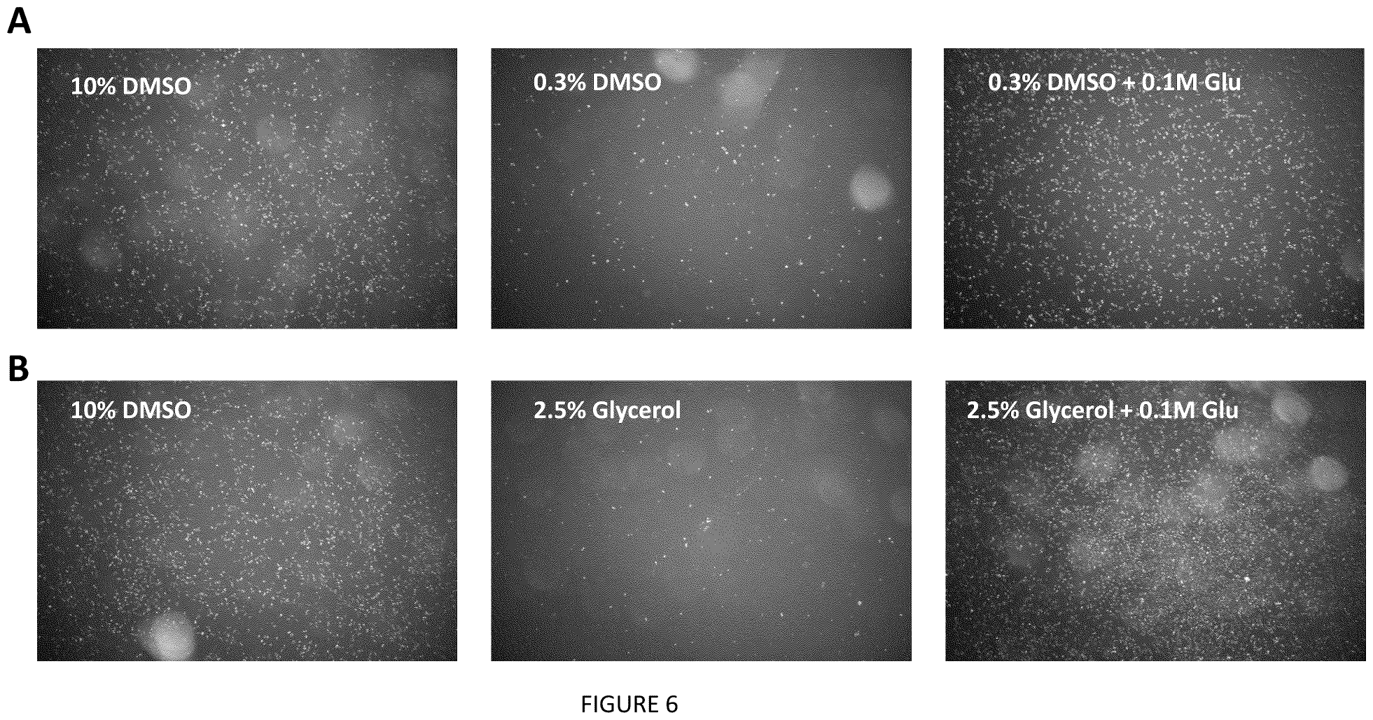

FIG. 6. Synergy of glutamate with DMSO and glycerol to create a new class of cryprotectant composition. Prior experiments demonstrated compatibility of glutamate with trehalose and betaine but without resolution to demonstrate if these molecules had synergy beyond potential additive effects. We determined whether glutamate had synergy with two better known cryoprotectants, DMSO and glycerol, by utilizing concentration of each that are known to have little or no cryprotective ability, e.g., 0.3%-1% DMSO and 2.5% glycerol. Thus, cryoprotectants that are at a concentration insufficient to provide cryoprotection when used alone can, for example, be understood to mean that cell survival is within 10% of control cell survival (for example, saline alone). Cells from adherent cultures prepared as described above were trypsinized, collected by centrifugation, and resuspended in saline. Data in panel A shows samples that were frozen in either 10% DMSO or 0.3% DMSO, as labeled in the top left of each photograph. A third sample was made 0.3% DMSO acentrifugation, andimilar set of samples was prepared in either 10% DMSO, 2.5% glycerol, and 2.5% glycerol in 0.1M glutamate, and is shown in panel B. From these data it is clear that the combination of glutamate and either cryoprotectant at this lower concentration yields recovery that is comparable to 10% DMSO.

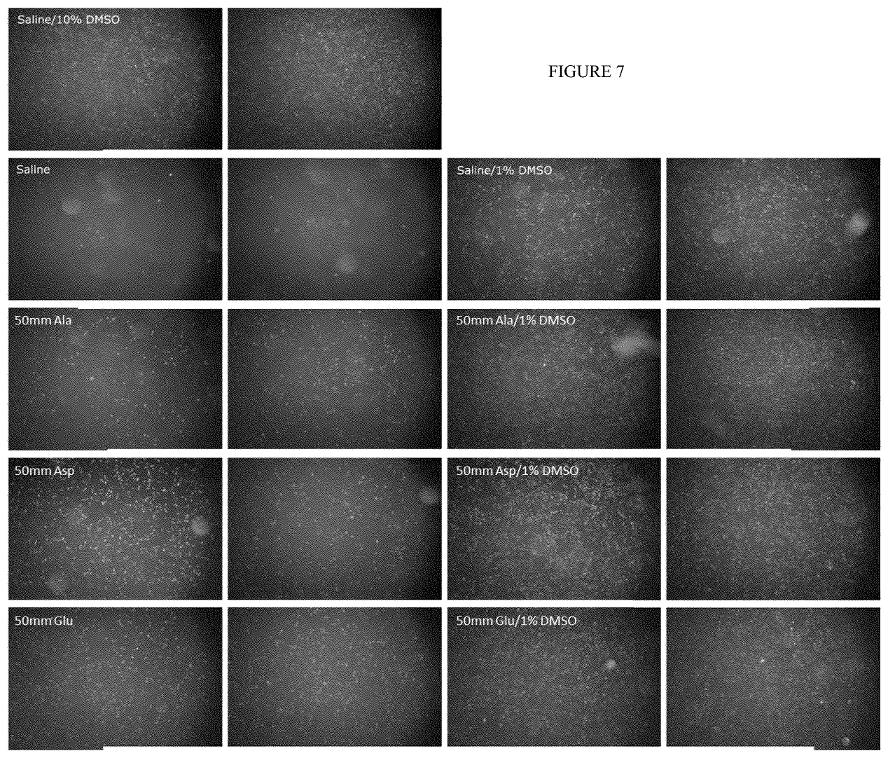

FIG. 7. Various single amino acids at 50 mM act synergistically with low DMSO concentrations to bring about effective cryoprotection. The ability of additional amino acids to serve as cryoprotectants beyond glutamate was tested in a study that utilized a number of single amino acids to act synergistically with low DMSO concentrations to bring about effective cryoprotection. The results are depicted in FIG. 7, with paired images (below center and center of each well) of cultures subjected to a freeze-thaw with the following cryoprotective formulations alone (columns 1 and 2) and with the addition of 1% DMSO (columns 3 and 4): 10% DMSO (row 1), saline (row 2), 50 mM alanine (row 3), 50 mM aspartate (row 4), and 50 mM glutamate (row 5).

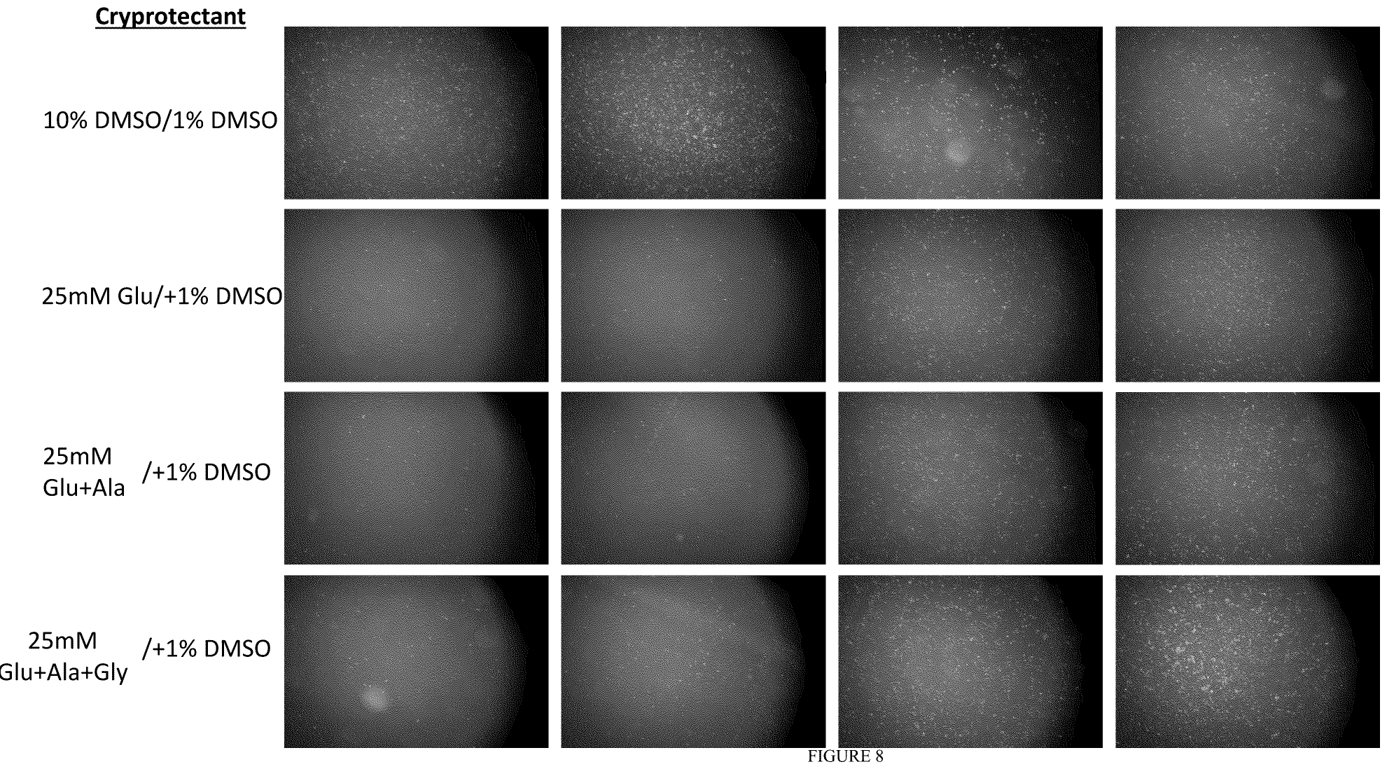

FIG. 8. Amino Acid mixtures exhibit enhanced synergy in the presence of low concentrations of DMSO. In order to test if amino acids can work synergistically in the presence of DMSO as well as its absence, Three formulations were compared for their ability to protect cells from freeze-thaw induced cytotoxicity: 25 mM glutamate, 25 mM of an equimolar mixture of glutamate+alanine, and 25 mM of an equimolar mixture of glutamate+alanine+glycine. The resulting data are shown in FIG. 8 as paired images, the first below center and the second near center for each sample. As labeled to the left of the figure, columns 1 and 2 corresponding to the formulation before the forward slash and columns 3 and 4 corresponding to the formulation after the slash, with “+1% DMSO” indicative of amino acid solution in the first two columns with the addition of 1% DMSO.

The studies shown in the data from FIGS. 2-6 demonstrate glutamate may have the properties of a very desirable cryoprotectant. In addition to its own biocompatibility, its activity is retained at lower concentrations by the addition of either (a) non-glutamate amino acids such as proline, threonine, or aspartate, or (b) addition of other known cryoprotectant such as DMSO or glycerol at concentrations that do not exhibit significant cryoprotection, but yield recovery levels similar to 10% DMSO. Another important property is the compatibility of glutamate as a cryoprotectant with physiological solutions such as growth medium, suggesting it may have broad applicability similar to dmso.

Various changes and modifications to the embodiments herein chosen for purposes of illustration will readily occur to those skilled in the art. To the extent that such modifications and variations do not depart from the spirit of the embodiments, they are intended to be included within the scope thereof.

### REFERENCES

1. KEITH S C Jr. Factors Influencing the Survival of Bacteria at Temperatures in the Vicinity of the Freezing Point of Water.Science. 1913 Jun. 6;37(962):877-9
2. PARKES A S. Preservation of Spermatozoa at Low Temperatures. Br Med J. 1945 Aug. 18;2(4415):212-3
3. MOLLISON P L, SLOVITER H A, CHAPLIN H Jr. Survival of transfused red cells previously stored for long periods in the frozen state. Lancet. 1952 Sep. 13;2(6733):501-5
4. SCHERER W F, HOOGASIAN A F. Preservation at subzero temperatures of mouse fibroblasts (strain L) and human epithelial cells (strain HeLa). Proc Soc Exp Biol Med. 1954 November;87(2):
5. LOVELOCK J E, BISHOP M W. Prevention of freezing damage to living cells by dimethyl sulphoxide. Nature. 1959 May 16;183(4672):1394-5
6. SALT, R W. Principles of Insect Cold-Hardiness. Ann Rev Ent. 1961; 6:55-74.
7. KAROW A M Jr. Cryoprotectants--a new class of drugs. J Pharm Pharmacol. 1969 April;21(4):209-23
8. ASHWOOD-SMITH M J. Mechanisms of cryoprotectant action. Symp Soc Exp Biol. 1987;41:395-406
9. ALVAREZ J G, STOREY B T. Evidence for increased lipid peroxidative damage and loss of superoxide dismutase activity as a mode of sublethal cryodamage to human sperm during cryopreservation. J Androl 1992; 13:232-241
10. LIMAYE L S. Bone marrow cryopreservation: improved recovery due to bioantioxidant additives in the freezing solution. Stem Cells. 1997;15(5):353-8
11. ZHANG L, XUE X, YAN J, YAN L Y, JIN X H, ZHU X H, HE Z Z, LIU J, LI R, QIAO J. L-proline: a highly effective cryoprotectant for mouse oocyte vitrification. Sci Rep. 2016Jul. 14;6:
12. ELLIOT G D, WANG S, FULLER B J. Cryoprotectants: A review of the actions and applications of cryoprotective solutes that modulate cell recovery from ultra-low temperatures. Cryobiology 76 (2017) 74 91
13. GAO L, ZHOU Q, ZHANG Y, SUN S, LV L, MA P, YANG J, LIU M, ZHANG L, WANG X, ZHAN L. Dimethyl Sulfoxide-Free Cryopreservation of Human Umbilical Cord Mesenchymal Stem Cells Based on Zwitterionic Betaine and Electroporation. Int. J. Mol. Sci. 2021, 22, 7445.

1. A cryopreservative composition, comprising an amino acid at a concentration between 50 mM and 1 M.

2. The composition of claim 1, wherein between 10-90% of volume comprises a base medium.

3. The composition of claim 2, wherein the base medium further comprises a serum-containing or serum-free culture medium.

4. The composition of claim 1, wherein said composition comprises glutamate and at least one additional amino acid, with a final cumulative amino acid concentration of between 100 mM and 1M.

5. The composition of claim 1, further comprising one or more of DMSO, glycerol, trehalose, and betaine.

6. The composition of claim 1, wherein said amino acid is glutamate at a concentration between 50mM and 1 M.

7. The composition of claim 6, wherein said composition further comprises at least one other amino acid with a final cumulative amino acid concentration of between 100 mM and 1M.

8. The composition of claim 6, further comprising one or more of DMSO, trehalose, and betaine.

9. A cryopreservative composition, comprising one or more amino acids and one or more cryoprotectants wherein said cryoprotectants are at a concentration insufficient to provide cryoprotection when used alone.

10. The composition of claim 9, wherein said one or more cryoprotectants comprise DMSO at a concentration of 1% or less.

11. The composition of claim 9, wherein said one or more cryoprotectants comprise glycerol at a concentration of 2.5% or less.

12. The composition of claim 10, wherein between 10-90% of volume comprises a base medium.

13. The composition of claim 12, wherein the base medium further comprises a serum containing or serum free cell culture medium.

14. The composition of claim 9, wherein said composition comprises an equimolar mixture of glutamate and alanine.

15. The composition of claim 9, wherein said composition comprises an equimolar mixture of glutamate, alanine, and glycine.

16. The composition of claim 9, wherein said cryoprotectants comprise one or more of DMSO, glycerol, trehalose, and betaine.

17. A method for cryopreservation of a cell, comprising the step of combining said cell with a cryopreservative composition comprising one or more amino acids and one or more cryoprotectants, wherein said cryoprotectants are at a concentration insufficient to provide cryoprotection when used alone.

18. The method of claim 17, wherein said one or more amino acids is an equimolar mixture of glutamate and alanine.

19. The method of claim 17, wherein said one or more cryoprotectants comprise DMSO at a concentration of 1% or less.

20. The method of claim 17, wherein said one or more cryoprotectants comprise glycerol at a concentration of 2.5% or less.
